(19) United States
(12) Patent Application Publication
(10) Pub. No.: US 2009/0030502 A1
(43) Pub. Date: Jan. 29, 2009

(54) Socket For Fenestrated Tubular Prosthesis

(76) Inventors: Jichao Sun, West Lafayette, IN (US); Alan R. Leewood, Lafayette, IN (US)

Correspondence Address: BRINKS HOFER GILSON & LIONE/CHICAGO/COOK, PO BOX 10395, CHICAGO, IL, 60610, US

(21) Appl. No.: 12/136,857

(22) Filed: Jun. 11, 2008

Related U.S. Application Data
(60) Provisional application No. 60/962,109, filed on Jul. 26, 2007.

(51) Int. Cl. A61F 2/06

(52) U.S. Cl. 623/1.16

(57) ABSTRACT

A stent graft adapted to telescopically receive a secondary stent graft characterized in that the stent graft comprises at least one socket communicating with at least one opening in the stent graft. The at least one socket comprises an elastic wall that forms a lumen with a stent at least partially encased within the wall. The socket can be adapted for use with stent grafts for implantantation in an aneurysm.

### CROSS REFERENCE TO RELATED APPLICATION

This application claims the benefit of U.S. Provisional Application No. 60/962,109, filed Jul. 26, 2007, which is incorporated by reference in its entirety.

### TECHNICAL FIELD

This invention relates to a medical device for implantation within the human or animal body for the treatment or repair of aortic aneurysms.

### BACKGROUND

One of the primary functions of the fenestrated stent graft with bridging stent is to maintain patency of the renal arteries even though the proximal end of the stent-graft extends beyond the renal arteries. Conventionally, a balloon expandable bare stent is deployed into the renal arteries through the fenestration in the main graft to assure alignment is maintained while the stent-graft is being delivered (e.g., manipulated) and continues to maintain patency post-procedure. Fenestrated stent grafts usually use a sutured nitinol ring with gold markers (see FIG. 1). The distal part of the metal stent is deployed into the renal artery and the proximal end is held against the graft via the sutured nitinol ring to ensure a secure fixation.

Since the arterial tree is constantly under pulsatile motion due to hemodynamic and anatomical loads, the deployed bare metal stent is very often under severe and complicated loading conditions (bending, radial pulsation, shearing, etc.) This must be borne entirely through the narrow interface presented by the nitinol ring. Furthermore, there is normally considerable plastic deformation induced to the stent during current deployment techniques which can lead to localized fracture of the stent that negates the alignment function of the fenestration stent.

The patency of the renal arteries may have to be maintained even though the proximal end of the stent-graft extends beyond the renal arteries. Conventionally, a balloon expandable bare stent is deployed into the renal arteries through a fenestration in the main graft to assure alignment is maintained while the stent-graft is being delivered. Current fenestrated stent grafts have a sutured nitinol ring with gold markers around the fenestration. The distal part of the metal stent is deployed into the renal artery or other branch vessel which the proximal end is secured to the fenestrated stent graft. The conventional fenestrated device and a deployed bare stent are shown in FIG. 1.

When an aneurysm extends infra-renally, a covered stent is needed to bridge this aneurysm so that the blood flow is maintained to the kidneys. In such cases, the interface between the fenestrated stent graft and the infra-renally placed covered stent must, in addition to providing alignment, provide a hemodynamic seal in a very dynamic environment. The difficulty in providing adequate renal support using either covered or bare metal stents is the narrow interaction zone between the infra-renally placed stent and the fenestrated stent graft. The infra-renally placed stent must handle the stresses caused by the pulsatile blood flow created by the heart.

One of the major functional requirements for an iliac branch vessel device bridging a covered stent is sealing and basic attachment. In order to achieve an effective seal at the proximal end where a covered stent fits into a Dacron bifurcated graft, devices in the art utilize two nitinol rings with a fixed diameter and a flexible stent with a nominal diameter less than the fixed diameter. However, due to the relative rigidity of the fixed diameter nitinol rings and the inextensibility of the Dacron graft, the socket can not exceed over about a mm from the fixed diameter. The resultant relatively rigid nature of this socket system restricts the proximal end of the bridging stent. Thus, the result is a stent which tapers along its axis, and very often non-uniformly as the bridging stent transitions out of the branch vessel device socket.

This raises two important issues: the effectiveness of the seal across the wide range of vessel sizes and the potential fatigue problems while undergoing pulsatile loading aggravated by the taper. A dramatic taper can potentially cause damaging plastic deformation and nonuniform loading on the covered Bx stent, especially within the transition region outside of the NiTi ring socket, which may greatly shorten stent durability or even tear or pinch the covering.

Further, since nitinol rings essentially create a fixed diameter socket, it will not accommodate the recoil of a covered stent. Therefore for some covered stents with a large recoil rate, the sealing function can be problematic.

Thus, a need exists for a socket for use with an endoluminal prosthesis which will minimize or eliminate the fatigue suffered by infra-renally placed stents. This would enable graft systems extending into renal arteries or other branched vessels to be safely utilized in patients for long periods of time without concern of premature failure due to wear. Such sockets need a high pulsatile fatigue life. Pulsatile fatigue is the fatigue resistance of the stent to pulsing radial loads, such as blood pressure loads. In practice, pulsatile fatigue is tested by expanding a stent into a flexible tube that is then filled with a fluid and pulsed rapidly to alter the diameter of the stent cyclically. Thus, a need exists for a prosthetic endovascular graft system which incorporates sockets that are designed to minimize cyclic stresses and thus avoid fatigue failure.

### BRIEF SUMMARY

The present invention provides a stent graft for endoluminal implantation. The stent graft is adapted to telescopically receive a secondary stent graft and is characterized in that the stent graft comprises at least one socket communicating with at least one opening in the stent graft. The at least one socket comprises an elastic wall that forms a lumen with a stent at least partially encased within the wall.

In another aspect of the invention, the stent graft is bifurcated with two distal openings and is adapted to telescopically receive a secondary stent graft. There is another aspect of the present invention wherein the socket forms a branch of the stent graft for telescopically receiving a secondary stent graft extending into an iliac artery. In another aspect, there is also a socket proximal to the bifurcation and comprises an elastic wall forming a lumen with a stent at least partially encased within the wall. In yet another aspect, the stent graft further comprises a second socket in communication with a second opening in the stent graft.

In one aspect of the present invention, the stent graft is adapted to telescopically receive a secondary stent graft extending into a renal artery. In yet another aspect, the proximal end of the socket flares around the external or internal side of the wall opening. The socket has an expandable diameter that adjusts to the dynamic movement of the human body. In some embodiments, the socket is tapered, comprises reinforcing elements, or radiopaque markers. The reinforcing elements comprise nitinol or polyethylene fibers. The socket can extend radially from the tubular prosthesis at an acute, right, or obtuse angle. There are also embodiments where the socket is attached to the tubular prosthesis by gluing, stitching, repolymerization, dipping, casting, or is thermoformed.

In yet another aspect of the present invention, the socket can be made from polyurethane, expanded polytetratfluoroethylene (ePTFE), or any other polymer that provides sufficient elasticity, deformability, and biocompatibility. Reinforcing elements, such as nitinol or PET fibers, may be imbedded in the socket to adjust the radial and longitudinal stiffness. Radiopaque markers, such as gold, can be placed within the socket to assist in placement of the socket.

In general, the stent grafts of the present invention provide sockets that have a high degree of expanded radial stiffness and flexibility which can be used for long periods of time in a pulsatile environment without causing fatigue and fracture of the socket or overall prosthesis. The sockets are highly torsional and distendable while bridging the tubular prosthesis and/or the structural prosthesis in the target vessel.

### BRIEF DESCRIPTION OF THE DRAWINGS

FIG. 1 is a close-up view of a bare stent protruding from a fenestrated stent graft found in the prior art.

FIG. 2 is a close-up view of a socket of the present invention attached to the wall and around the opening of a fenestrated stent graft.

FIG. 3 is a drawing of a socket of the present invention with a secondary stent telescopically placed within the socket.

FIG. 4 is a view of the proximal end of a stent graft of the present invention with a socket in communication with an opening in the stent graft.

FIG. 5 depicts a bifurcated stent graft with a socket portion in communication with a branch opening.

FIG. 6 illustrates a bifurcated stent graft with a socket with a secondary stent graft implanted into the socket.

FIGS. 7A through 7D are cross-sectional views of different embodiments of the sockets of the present invention.

FIG. 8 is a cut-away view of an abdominal aortic aneurysm with a stent graft of the present invention implanted in the aorta with sockets bridging secondary stent grafts implanted in the renal arteries.

FIG. 9 is a cut-away view of an abdominal aortic aneurysm with a stent graft of the present invention implanted in the aorta with a socket implanted into the iliac artery.

FIG. 10 depicts a branched prosthesis implanted in the aortic arch with sockets extending into branch arteries with one socket receiving a secondary stent graft.

### DETAILED DESCRIPTION OF THE DRAWINGS AND THE PREFERRED EMBODIMENTS

Unless defined otherwise, all technical and scientific terms used herein have the same meaning as commonly understood to one of ordinary skill in the art to which this invention belongs.

Throughout this specification, when discussing the application of this invention to the aorta, the term distal, with respect to a prosthesis, is intended to refer to the end of the prosthesis furthest away in the direction of blood flow from the heart, and the term proximal is intended to mean the end of the prosthesis that, when implanted, would be nearest to the heart.

The term “graft or graft material” means a generally cannular or tubular member which acts as an artificial vessel or prosthesis. A graft by itself or with the addition of other elements, such as structural components, can be an endoluminal prosthesis. The graft comprises a single material, a blend of materials, a weave, a laminate, or a composite of two or more materials. The graft can also comprise polymer material that may be layered onto the mandrel of the present invention. Preferably, polymers of the present invention, although added in layers onto the mandrel, after curing, result in one layer that encapsulates a stent or woven graft. This also aids in decreasing the incidence of delamination of the resulting endovascular prosthesis.

The graft material is a biocompatible material that is both flexible and abrasion resistant. Furthermore, the graft material should be selected from those materials that are particularly well suited for bonding with polymer. Preferably, the graft material is a woven polyester. More preferably, the graft material is a polyethylene terephthalate (PET), such as DACRON® (DUPONT, Wilmington, Del.) or TWILLWEAVE MICREL® (VASCUTEK, Renfrewshire, Scotland). Woven polyesters, such as Dacron, possess varying degrees of porosity, where the degree of porosity can be selectively controlled based on the weaving or knitting process that is used to produce the woven polyester. Consequently, depending on the application, the porosity can be adjusted to encourage incorporation of a patient's tissue into the woven graft material, which in turn may more securely anchor the prosthesis within the patient's vessel or lumen. Furthermore, the degree of porosity can also be adjusted to provide a woven graft material that is impermeable to liquids, including blood or other physiological fluids.

In another embodiment, the woven graft material may be made of a single material, or it may be a blend, weave, laminate, or composite of two or more materials. The graft material may also include other additives, such as plasticizers, compatibilizers, surface modifiers, biological materials such as peptides and enzymes, and therapeutic agents such as drugs or other pharmaceutically effective medicaments. The therapeutic agents can comprise agents, or combinations thereof, that can affect the cells in a vessel wall, including drugs, chromophores, and nucleic acids. Therapeutic agents also comprise diagnostics such as radiopaque compounds that allow the vessel to be visualized by fluoroscopy or like methods. Therapeutic agents can also comprise antimicrobial agents, such as antibacterial and antiviral agents.

It may be preferred that the socket includes a biocompatible polyurethane. Examples of biocompatible polyurethanes include Thoralon® (THORATEC, Pleasanton, Calif.), BIOSPAN®, BIONATE®, ELASTHANE®, PURSIL® and CARBOSIL® (POLYMER TECHNOLOGY GROUP, Berkeley, Calif.). As described in U.S. Pat. Pub. No. 2002/0065552 A1, incorporated herein by reference, Thoralon® is a polyetherurethane urea blended with a siloxane-containing surface modifying additive. Specifically, the polymer is a mixture of base polymer BPS-215 and an additive SMA-300. The concentration of additive may be in the range of 0.5% to 5% by weight of the base polymer. The BPS-215 component (THORATEC) is a segmented polyether urethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED). The SMA-300 component (THORATEC) is a polyurethane comprising polydimethylsiloxane as a soft segment and the reaction product of MDI and 1,4-butanediol as a hard segment. A process for synthesizing SMA-300 is described, for example, in U.S. Pat. Nos. 4,861,830 and 4,675,361, which are incorporated herein by reference. A polymer graft material can be formed from these two components by dissolving the base polymer and additive in a solvent such as dimethylacetamide (DMAC) and solidifying the mixture by solvent casting or by coagulation in a liquid that is a non-solvent for the base polymer and additive.

Thoralon® has been used in certain vascular applications and is characterized by thromboresistance, high tensile strength, low water absorption, low critical surface tension, and good flex life. Thoralon® is believed to be biostable and to be useful in vivo in long term blood contacting applications requiring biostability and leak resistance. Because of its flexibility, Thoralon® is useful in larger vessels, such as the abdominal aorta, where elasticity and compliance is beneficial.

Other polyurethane ureas may be used in addition to Thoralon. For example, the BPS-215 component with a MDI/PTMO mole ratio ranging from about 1.0 to about 2.5 may be used.

In addition to polyurethane ureas, other polyurethanes, preferably those having a chain extended with diols, may be used as the graft material. Polyurethanes modified with cationic, anionic, and aliphatic side chains may also be used. See, for example, U.S. Pat. No. 5,017,664, which is incorporated herein by reference. Polyurethanes may need to be dissolved in solvents such as dimethyl formamide, tetrahydrofuran, dimethyacetamide, dimethyl sulfoxide, or mixtures thereof.

The polyurethanes may also be end-capped with surface active end groups, such as, for example, polydimethylsiloxane, fluoropolymers, polyolefin, polyethylene oxide, or other suitable groups. See, for example, the surface active end groups disclosed in U.S. Pat. No. 5,589,563, which is incorporated herein by reference.

In one embodiment, the graft material may contain a polyurethane having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. Examples of siloxane-polyurethanes are disclosed in U.S. Pat. Pub. No. 2002/0187288 A1, which is incorporated herein by reference.

The graft may contain polytetrafluoroethylene or ePTFE. The structure of ePTFE can be characterized as containing nodes connected by fibrils. The structure of ePTFE is disclosed, for example, in U.S. Pat. Nos. 6,547,815 B2; 5,980,799; and 3,953,566; all of which are incorporated herein by reference.

If so desired, the polymers described above can be processed to form porous polymer grafts using standard processing methods, including solvent-based processes such as casting, spraying, dipping, melt extrusion processes, repolymerization or thermoformation. Extractable pore forming agents can be used during processing to produce porous polymer graft material. Examples of the particulate used to form the pores include a salt, including, but not limited to, sodium chloride (NaCl), sodium bicarbonate (NaHCO₃), Na₂CO₃, MgCl₂, CaCO₃, calcium fluoride (CaF₂), magnesium sulfate (MgSO₄), CaCl₂, AgNO₃, or any water soluble salt. However, other suspended particulate materials may be used. These include, but are not limited to, sugars, polyvinyl alcohol, cellulose, gelatin, or polyvinyl pyrolidone. Preferably, the particulate is sodium chloride; more preferably, the particulate is a sugar.

Therapeutic agents can be incorporated into the graft material of the prosthesis, or into the biocompatible coating which encapsulates the stent, so that they can be released into the body surrounding the lumen wall upon expansion and curing of the prosthesis. Therapeutic agents or medicaments can be impregnated into the lumen wall by pressure from expansion of the prosthesis. The therapeutic agent can also be photoreleasably linked to the surface of the prosthesis so that, upon contact with the surrounding lumen wall, the agent is released onto the cells of the adjacent vascular wall by exposure to radiation delivered via an optical fiber.

The term “stent” means any device that provides rigidity, expansion force, or support to a prosthesis, such as a stent graft. In one configuration, the stent may represent a plurality of discontinuous devices. In another configuration, the stent may represent one device. The stent may be located on the exterior of the device, the interior of the device, or both. Stents may have a wide variety of configurations and may be balloon-expandable or self-expanding. Typically, stents have a circular cross-section when fully expanded, so as to conform to the generally circular cross-section of a body lumen. In one example, a stent may comprise struts and acute bends or apices that are arranged in a zig-zag configuration in which the struts are set at angles to each other and are connected by the acute bends. The stent struts may have a thickness ranging from about 0.060 mm to about 0.20 mm and all combinations and subcombinations therein.

Preferably, the stent is formed from nitinol, stainless steel, tantalum, titanium, gold, platinum, inconel, iridium, silver, tungsten, cobalt, chromium, or another biocompatible metal, or alloys of any of these. Examples of other materials that may be used to form stents include carbon or carbon fiber; cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, polytetrafluoroethylene, or another biocompatible polymeric material, or mixtures or copolymers of these; polylactic acid, polyglycolic acid or copolymers thereof; a polyanhydride, polycaprolactone, polyhydroxybutyrate valerate or another biodegradable polymer, or mixtures or copolymers of these; a protein, an extracellular matrix component, collagen, fibrin, or another biologic agent; or a suitable mixture of any of these. Preferably, the stent is a nitinol or stainless steel stent.

The socket may be comprised of biocompatible polyurethane, silicone infused polyurethane, such as Thoralon® (Thoratec, Pleasanton, Calif.), or Biospan®, Bionate®, Elasthane®, Pursil® And Carbosil® (Polymer Technology Group, Berkeley, Calif.). In some embodiments, polyurethane can also comprise SIS. The sockets may comprise a single biologically active material or a blend of materials that are thromboresistant. The sockets are thromboresistant without the addition of foams, adhesives, or polymers. In embodiments that may be preferred, the sockets are attached to the stent graft by reploymerization or they are thermoformed to the stent grafts.

Thoralon® has been used in certain vascular applications and is characterized by thromboresistance, high tensile strength, low water absorption, low critical surface tension, and good flex life. Thoralon® is believed to be biostable and to be useful in vivo in long term blood contacting applications requiring biostability and leak resistance. Because of its flexibility, Thoralon® is useful in larger vessels, such as the abdominal aorta, where elasticity and compliance is beneficial.

The stent graft of the present invention has a stent graft wall 20 comprising a graft material for endoluminal implantation. As seen in FIG. 2, the stent graft wall 20 has an opening 25 that, once the stent graft is deployed, should align with a branch vessel. At least one elastic, deformable socket 30 is in communication with the opening 25. The socket 30 extends from the stent graft wall 20 of the stent graft with shock absorbing and elastic properties. The socket comprises a socket wall having a proximal end 23 and a distal end 29 with the proximal end 23 interconnected with the stent graft wall 20. The proximal end 23 of the socket 30 surrounds the opening 25 and the distal end 29 extends in a distal direction from the stent graft wall 20. The wall of the socket 30 at least partially encases a stent 27.

There are other embodiments further comprising a secondary socket for receiving a secondary stent graft comprising an elastic wall forming a lumen. Some embodiments have at least one opening and at least one socket with the at least one socket being in communication with the openings. In some embodiments, that may be preferred, the stent graft further comprises a secondary socket that has no stent encased within the secondary socket wall. Such an embodiment would have more than one socket attached to the stent graft having a stent encased within its walls and a secondary socket with no encased stent. Any of the sockets are capable of telescopically receiving a secondary stent graft.

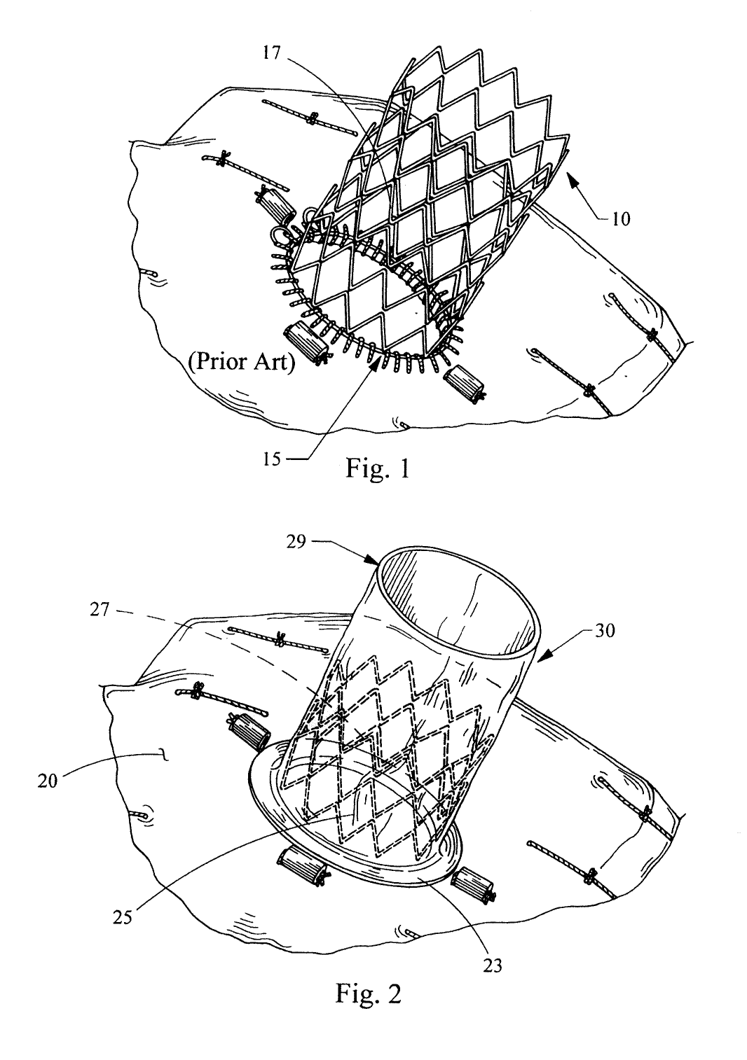

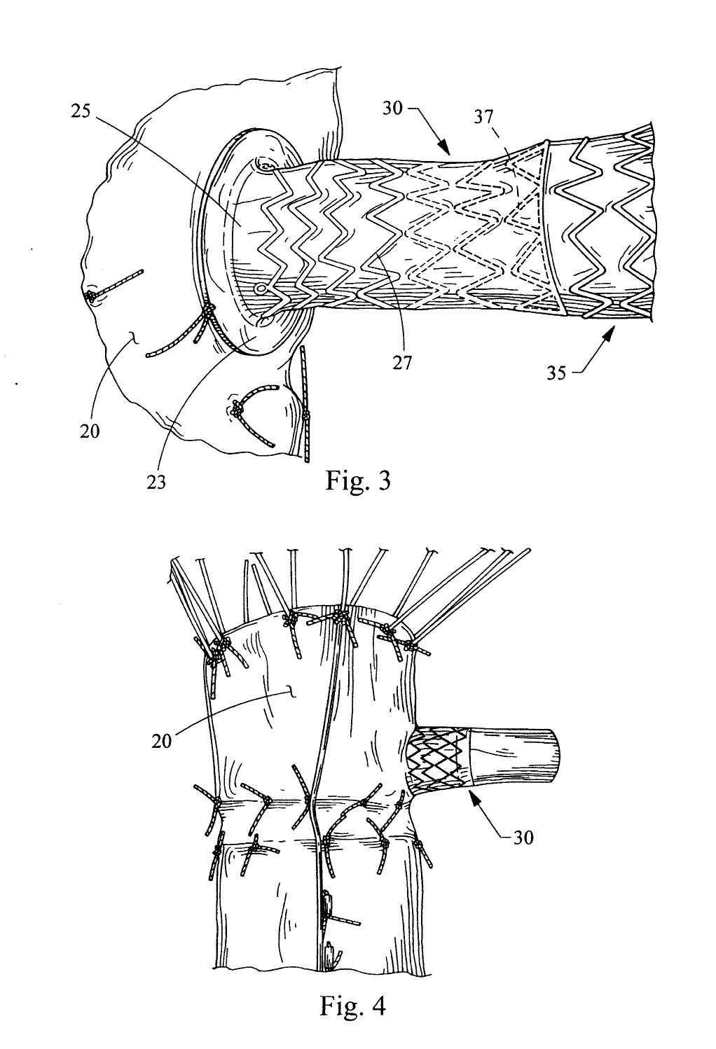

FIG. 1 is an illustration of a socket 10 commonly used in the prior art containing a stent 17 attached to a nitinol ring 15 sewn around the opening 25 in the graft. As seen in FIGS. 2 and 3, the proximal end 23 of the socket 30 of the present invention flares around the opening 25 in the stent graft wall 20. Although the proximal end of the socket shown flares around the external side of the graft wall, there are embodiments where the socket flares around the internal side of the graft wall. Due at least in part to its elastic attributes, the socket 30 has an expandable diameter. The present invention provides at least one socket 30 that may be attached to the stent graft wall 20 by repolymerization. For instance, a solvent such as dimethylacetamide (DMAC) is used to partially dissolve the polymer such that is penetrates into the graft material of the stent graft wall 20. The polymer is then allowed to repolymerize. In some embodiments, the socket 30 may be glued to the stent graft wall 20. The polymer and DMAC can be used together in solution as a glue to attach the socket 30 and stent graft wall 20. In other embodiments, dipping or casting can be used to join the socket 30 and stent graft wall 20. Preferably, the means of attachment provides the socket 30 with a hermetic seal with the stent graft wall 20.

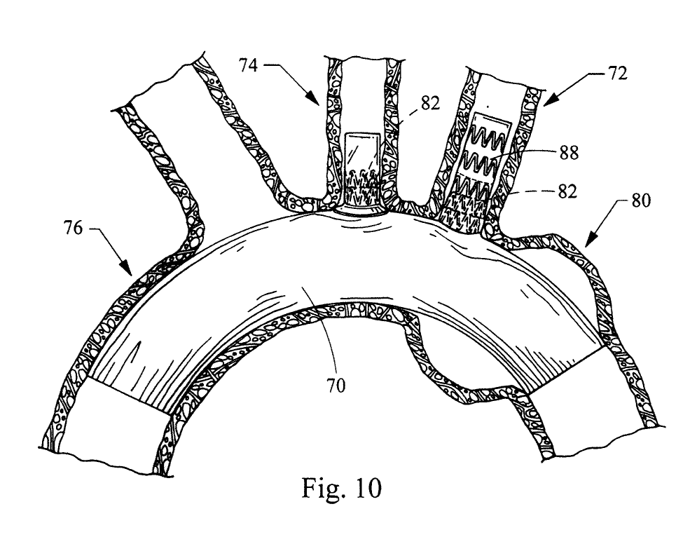

Some embodiments that may be preferred provide a branched prosthesis for implantation in an aortic arch 76 as shown in FIG. 10. The stent graft wall of the prosthesis 70 spans the aortic arch 76 and has two elastic, deformable sockets 82 deployed in the carotid 74 and subclavian 72 arteries. A stent graft 88 is shown deployed in the socket 82 within the subclavian artery 72. Although not shown, there are embodiments having at least one socket or up to three sockets, one for each branch artery.

There are embodiments of the present invention that provide a compliant connection between a branched prosthesis and a stent or stent-graft deployed in the iliac or renal arteries. This may help to lower the peak loads transferred through the interface and better accommodate the very dynamic nature of the operating environment.

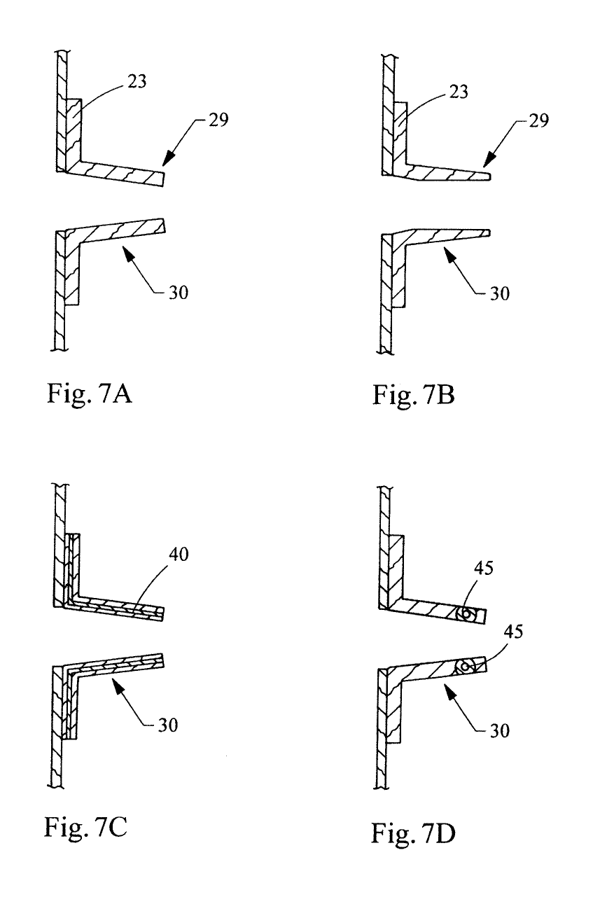

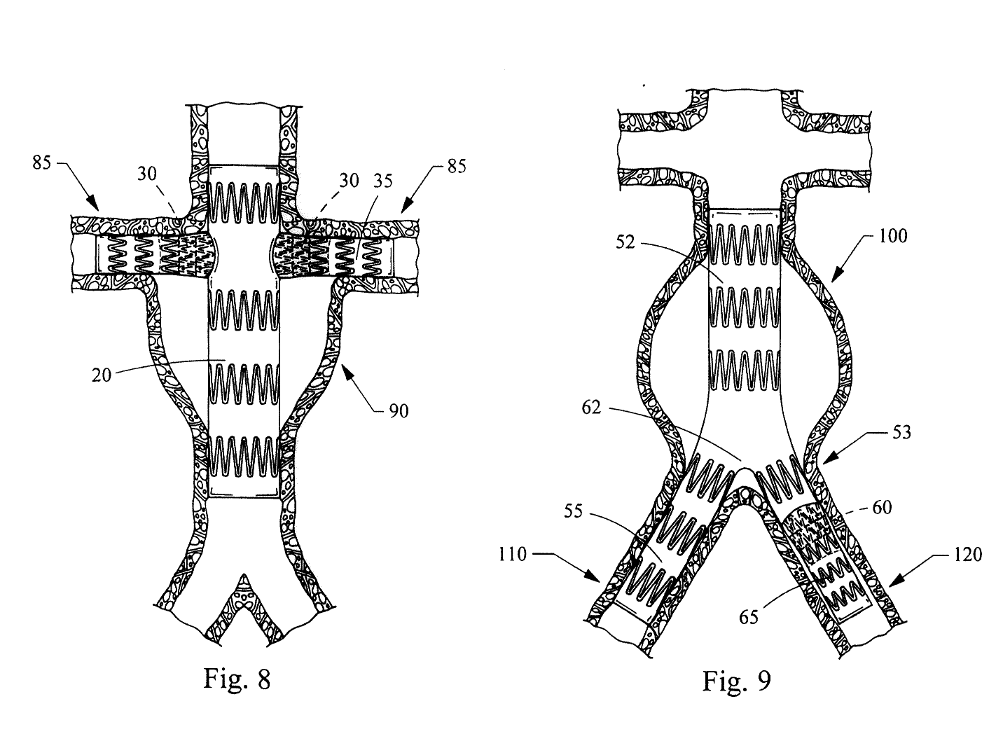

FIGS. 7A through 7D show cross-sectional views of the socket 30. In FIG. 7A, the illustration shows a cross-sectional view of a socket 30 tapering off to a diameter smaller at the distal end 29 than at the proximal end 23. Such a design may facilitate tracking during deployment and also help secure the stent and maintain sealing. This will also allow the radial stiffness of the socket 30 to vary along its length which will allow designs to span a wider variation in diameter without providing excessive force which may tend to crush other stents in the art.

The thickness and length of the socket 30 may vary, as in FIG. 7B, so that its radial stiffness can be controlled. The radial stiffness of the socket 30 may impact sealing and the pull out force. The socket 30 extends radially from the stent graft wall at an acute, right, or obtuse angle in varying embodiments. The socket 30 can comprise Thoralon which demonstrates significant elasticity (approximately 900%) so as to provide a wide range of operation. Alternatively, one skilled in the art can see a wide range of materials may be used herein, which includes ePTFE, polyurethane, and any other polymers which exhibit sufficient elasticity and/or deformability and, of course, biocompatibility.

Some embodiments of the socket 30 comprise reinforcing elements. These elements can comprise nitinol, stainless steel, or polyethylene fibers, for example. FIG. 7C shows a socket 30 encasing a reinforcing element 40. In some embodiments, the socket 30 comprises radiopaque markers 45. In the embodiment illustrated in FIG. 7D, the markers 45 are in the distal end 29 of the socket 30. Gold markers, for instance, can be embedded within the socket 30 to ensure accurate deployment.

The socket 30 can have reinforcing elements, such as nitinol or PET fibers, imbedded to alter the radial and longitudinal stiffness of the socket 30, as shown in FIG. 7C. The resultant composite socket 30 can limit the range of its motion as a function of stent design. Therefore, the ultimate diameter of the socket 30 can be controlled to help prevent possible excessive vessel damage as the embedded reinforcing elements can be used to limit the expansion of the socket 30 during stent deployment. A stent can be a reinforcing element. Socket 30 stiffness can be adjusted to the different radial stiffness exhibited in self-expanding stents.

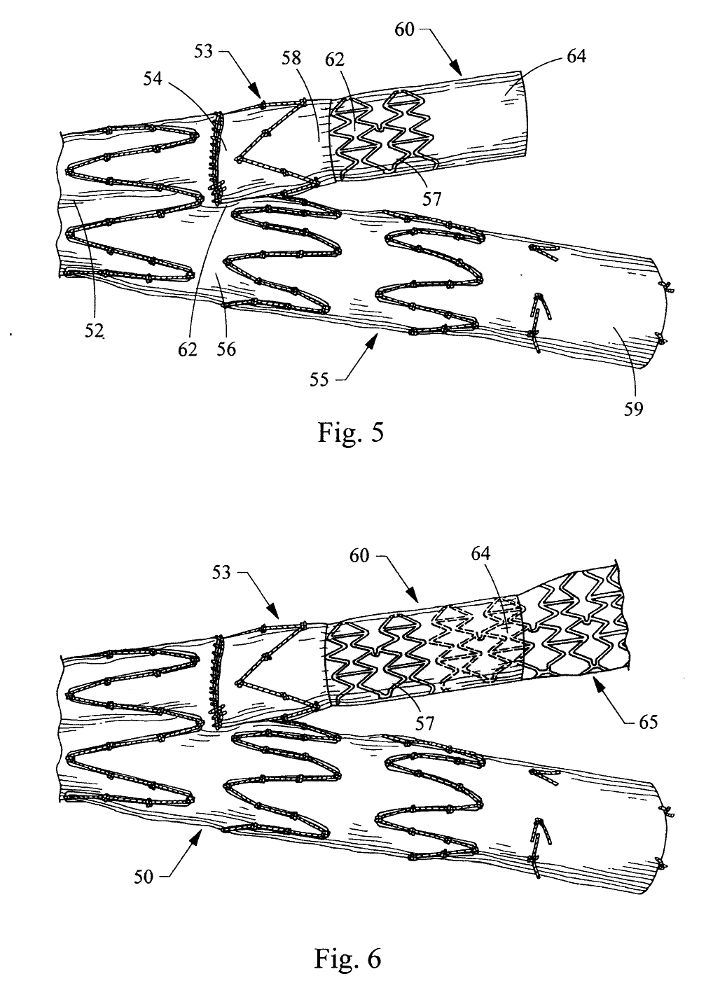

As illustrated in FIGS. 5 and 6, another embodiment of the present invention provides a bifurcated stent graft 50 with two distal openings for deployment in the abdominal aorta. This stent graft 50 comprises a main section 52 that forms a main lumen configured for deployment in the aorta. There is a first branch section 53 and a second branch section 55 both having proximal portions 54, 56 and distal portions 58, 59. The proximal portion 54 of the first branch section 53 and the proximal portion 56 of the second branch section 55 meet with the main section 52 at the bifurcation 62. The first branch 53 and second branch 55 sections comprise a graft material and are configured for deployment in vessels arteries branching from aorta. The first branch 53 and second branch 55 sections forming a first lumen and a second lumen, respectively. The lumens are in fluid communication with the main lumen of the main section 52.

The bifurcated stent graft 50 further comprises an elastic, deformable socket 60 in communication with the opening at the distal portion 58 of the first branch section 53. The socket 60 comprises a socket wall that forms a socket lumen with the socket 60 having a proximal end 62 and a distal end 64. The proximal end 62 being attached to the graft material of a branch section at the distal portion of that branch section. Although the socket 60 shown is attached with the distal portion 58 of the first branch section 53, there are also embodiments not shown wherein a socket is attached with the distal portion 59 of the second branch 55 section. It is understood then that the opening of the stent graft in such embodiments is in the distal portions 58, 59.

The elastic, deformable sockets of the present invention are configured to be receptive to tubular prostheses suitable for deployment in branch vessels. FIG. 3 is a close-up view of a secondary stent graft 35 deployed in the distal end of the deformable socket 30 in one particular embodiment. The stents 37 of the secondary stent graft 35 are indicated with dashed markings. Such an embodiment is suitable for implantation in an abdominal aortic aneurysm (AAA)

1. A stent graft for endoluminal implantation, wherein the stent graft is adapted to telescopically receive a secondary stent graft, characterized in that the stent graft comprises at least one socket communicating with at least one opening in the stent graft, the at least one socket comprising an elastic wall forming a lumen with a stent at least partially encased within the wall.

2. The stent graft of claim 1 wherein the at least one socket comprises a proximal end and the proximal end flares around the at least one opening in the stent graft.

3. The stent graft of claim 1 wherein the at least one socket has an expandable diameter.

4. The stent graft of claim 1 wherein the at least one socket is tapered.

5. The stent graft of claim 1 wherein the at least one socket further comprises reinforcing elements comprising nitinol or polyethylene fibers.

6. The stent graft of claim 1 wherein the at least one socket extends radially from the stent graft at an acute, right, or obtuse angle.

7. The stent graft of claim 1 wherein the at least one socket is attached to the stent graft by repolymerization or is thermoformed.

8. The stent graft of claim 1 wherein the at least one socket comprises radiopaque markers.

9. The stent graft of claim 1 wherein the at least one socket is comprised of polyurethane or ePTFE.

10. The stent graft of claim 1 wherein the stent graft is suitable for placement in an abdominal aortic aneurysm or a thoracic aortic aneurysm.

11. The stent graft of claim 1 wherein the at least one opening corresponds to a renal artery and the at least one socket is adapted for receiving a secondary stent graft extending into a renal artery.

12. The stent graft of claim 1 further comprising a second socket in communication with a second opening in the stent graft.

13. The stent graft of claim 1 wherein the stent graft further comprises a reinforcing ring around the opening and the stent is attached to the reinforcing ring while at least partially encased within the wall of the at least one socket.

14. The stent graft of claim 1 wherein the stent graft is adapted for placement in the aortic arch.

15. The stent graft of claim 1 wherein the at least one opening corresponds to a branch artery in the aortic arch and the at least one socket is adapted for receiving a secondary stent graft extending into the branch artery.

16. The stent graft of claim 1 comprising two sockets.

17. The stent graft of claim 1 further comprising a secondary socket for receiving a secondary stent graft comprising an elastic wall forming a lumen.

18. A stent graft for endoluminal implantation, wherein the stent graft is bifurcated with two distal openings and is adapted to telescopically receive a secondary stent graft, characterized in that the stent graft comprises at least one socket communicating with at least one opening in the stent graft, the at least one socket comprising an elastic wall forming a lumen with a stent at least partially encased within the wall and is adapted to telescopically receive a secondary stent graft.

19. The stent graft of claim 18 further comprising a socket proximal to the bifurcation, the socket comprising an elastic wall forming a lumen with a stent at least partially encased within the wall.

20. The stent graft of claim 18 wherein the at least one socket telescopically receives a secondary stent graft extending into an iliac artery.

21. The stent graft of claim 18 wherein the at least one socket has an expandable diameter.

22. The stent graft of claim 18 wherein the at least one socket is tapered.

23. The stent graft of claim 18 wherein the at least one socket further comprises reinforcing elements comprising nitinol or polyethylene fibers.

24. The stent graft of claim 18 wherein the at least one socket is attached to the stent graft by repolymerization or is thermoformed.

25. The stent graft of claim 18 wherein the at least one socket comprises radiopaque markers.

26. The stent graft of claim 18 wherein the at least one socket is comprised of polyurethane or ePTFE.

27. The stent graft of claim 18 comprising two sockets.

28. A stent graft for endoluminal implantation wherein the stent graft is adapted to telescopically receive a secondary stent graft extending into a renal artery, characterized in that the stent graft comprises at least one socket communicating with at least one opening in the stent graft, wherein at least one socket comprises a proximal end that flares around the at least one opening and an elastic wall forming a lumen with a stent at least partially encased within the wall.

29. The stent graft of claim 28 wherein the at least one socket comprises a proximal end and the proximal end flares around the at least one opening in the stent graft.

30. The stent graft of claim 28 wherein the at least one socket has an expandable diameter.

31. The stent graft of claim 28 wherein the at least one socket is tapered.

32. The stent graft of claim 28 wherein the at least one socket further comprises reinforcing elements comprising nitinol or polyethylene fibers.

33. The stent graft of claim 28 wherein the at least one socket extends radially from the stent graft at an acute, right, or obtuse angle.

34. The stent graft of claim 28 wherein the at least one socket is attached to the stent graft by repolymerization or is thermoformed.

35. The stent graft of claim 28 wherein the at least one socket comprises radiopaque markers.

36. The stent graft of claim 28 wherein the at least one socket is comprised of polyurethane or ePTFE.

37. The stent graft of claim 28 further comprising a secondary socket in communication with a second opening in the stent graft.

38. The stent graft of claim 28 further comprising a secondary socket for receiving a secondary stent graft comprising an elastic wall forming a lumen.

39. The stent graft of claim 28 wherein the stent graft further comprises a reinforcing ring around the opening and the stent is attached to the reinforcing ring while at least partially encased within the wall of the at least one socket.
